(19) United States
(12) Patent Application Publication
(10) Pub. No.: US 2007/0225709 A1
(43) Pub. Date: Sep. 27, 2007

(54) SINGLE PRONG IN SITU SPREADER

(76) Inventor: Mark H. Falahee, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/690,489

(22) Filed: Mar. 23, 2007

Related U.S. Application Data
(60) Provisional application No. 60/785,219, filed on Mar. 23, 2006.

(51) Int. Cl. A61F 2/30

(52) U.S. Cl. 606/61

(57) ABSTRACT

Spinal instrumentation and methods are adapted for use with implanted pedicle screws having exposed heads with opposing surfaces between a bony surface and a rod-capturing through bore. The instrumentation includes a spreader having a proximal end and a distal end, the proximal end including a pair of handles and the distal end including a single pair prongs. The prongs of the spreader have tapered ends with opposing surfaces adapted to engage with the opposing surfaces of the exposed heads of the pedicle screws and spread them apart when pressure is applied to the handles. A method of spreading implanted pedicle screws having exposed heads, comprises the steps of inserting the prongs of the spreader between the opposing surfaces of the exposed pedicle screw heads, and spreading the exposed heads through the application of pressure to the handles of the spreader. The method may further including the step of applying a distractor over the prongs of the spreader when the exposed pedicle screw heads are moved apart.

### REFERENCE TO RELATED APPLICATION

This application claims priority from U.S. Provisional Patent Application Ser. No. 60/785,219, filed on Mar. 23, 2006, the entire content of which is incorporated herein by reference.

### FIELD OF THE INVENTION

This invention relates generally to spinal surgery and, in particular, to an in situ spreader utilized to move apart adjacent pedicle screw heads, with or without a rod.

### BACKGROUND OF THE INVENTION

As shown in FIG. 1, current surgical spreaders are double-pronged, as they are designed to engage upper and lower surfaces of a rod between the pedicle heads to facilitate spreading. However, in many instances, the pedicle heads are very close together, if not touching, making it impossible to use a double-pronged spreader in these situations. The problem is usually that the top prong cannot pass between the converging heads, whether or not a rod has been used. Moreover, when utilizing a distractor to spread open the disc space for TLIF or PLIF procedures, the close proximity of the heads makes engaging the distractor cumbersome, if not impossible. As shown in FIG. 2, even if a double-pronged spreader can somehow be wedged into place between the screw heads, the upper prong of the spreader still interferes with the distractor.

### SUMMARY OF THE INVENTION

Spinal instrumentation and methods are adapted for use with implanted pedicle screws having exposed heads with opposing surfaces between a bony surface and a rod-capturing through bore. The instrumentation includes a spreader having a proximal end and a distal end, the proximal end including a pair of handles and the distal end including a single pair prongs. The prongs of the spreader have tapered ends with opposing surfaces adapted to engage with the opposing surfaces of the exposed heads of the pedicle screws and spread them apart when pressure is applied to the handles.

A method of spreading implanted pedicle screws having exposed heads, comprises the steps of inserting the prongs of the spreader between the opposing surfaces of the exposed pedicle screw heads, and spreading the exposed heads through the application of pressure to the handles of the spreader. The method may further including the step of applying a distractor over the prongs of the spreader when the exposed pedicle screw heads are moved apart.

### BRIEF DESCRIPTION OF THE DRAWINGS

FIG. 1 is a drawing that shows problems associated with the use of current double-pronged spreaders;

FIG. 2 is a drawing which shows how the use of prior-art double-pronged spreaders interferes with the application of a distractor;

FIG. 3 is a drawing which shows a single-pronged spreader according to the invention; and

FIG. 4 shows the way in which a single-pronged spreader according to the invention is used.

### DETAILED DESCRIPTION OF THE PREFERRED EMBODIMENTS

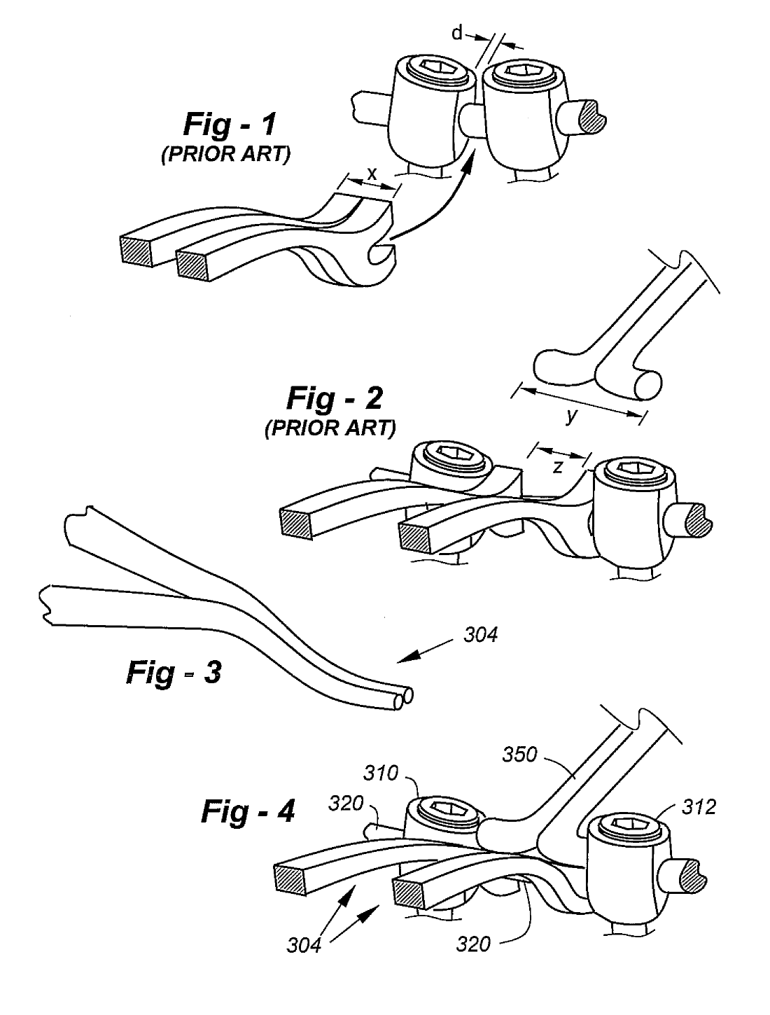

Having discussed the prior art with reference to FIGS. 1 and 2, the reader's attention is now turned to FIG. 3, which shows a single-pronged spreader according to this invention. The instrument has a proximal portion (not shown) with handles, the shape of which may be varied to include finger holes, and the like, with a distal portion including a pair of opposing prongs 304 having tapered ends.

As shown in FIG. 4, this allows the prongs to be inserted between pedicle screw heads 310, 312, typically against the outer surfaces of the screw heads where they emerge from the bone surface. The tapered ends of the spreader are dimensioned to fit below the rod 320, if present. As can be seen in the figure, this allows the pedicle screws to be spread apart (with the rod loosened), allowing more room for the distractor 350 to be subsequently applied.

I claim:

1. Spinal instrumentation adapted for use with implanted pedicle screws having exposed heads with opposing surfaces between a bony surface and a rod-capturing through bore, the instrumentation comprising:
a spreader having a proximal end and a distal end, the proximal end including a pair of handles and the distal end including a single pair prongs,
the prongs having tapered ends with opposing surfaces adapted to engage with the opposing surfaces of the exposed heads of the pedicle screws and spread them apart when pressure is applied to the handles.

2. A method of spreading implanted pedicle screws having exposed heads, comprising the steps of:
providing the instrumentation of claim 1;
inserting the prongs of the spreader between the opposing surfaces of the exposed pedicle screw heads; and
spreading the exposed heads through the application of pressure to the handles of the spreader.

3. The method of claim 2, further including the step of applying a distractor over the prongs of the spreader when the exposed pedicle screw heads are moved apart.
